(19) United States
(12) Patent Application Publication
(10) Pub. No.: US 2021/0143327 A1
(43) Pub. Date: May 13, 2021

(54) ORGANIC LIGHT-EMITTING COMPOUND AND ORGANIC ELECTROLUMINESCENT DEVICE USING THE SAME

(71) Applicant: DOOSAN SOLUS CO., LTD., Iksan-si, Jeollabuk-do (KR)

(72) Inventors: Jae Yi SIM, Yongin-si (KR); Min Sik EUM, Yongin-si (KR); Woo Jae PARK, Yongin-si (KR)

(73) Assignee: DOOSAN SOLUS CO., LTD., lksan-si, Jeollabuk-do (KR)

(21) Appl. No.: 17/047,797

(22) PCT Filed: Feb. 13, 2019
(86) PCT No.: PCT/KR2019/001758

§ 371 Date: Oct. 15, 2020

(30) Foreign Application Priority Data
Apr. 16, 2018 (KR) 10-2018-0043854

(51) Int. Cl. H01L 51/00; C07C 211/58; C09K 11/06; C07C 211/61; C07D 221/20; C07D 307/91; C07D 405/04; C07D 333/76; C07D 409/04

(52) U.S. Cl. CPC H01L 51/006; C07C 211/58; C09K 11/06; C07C 211/61; C07D 221/20; H01L 51/5088; C07D 307/91; C07D 405/04; C07D 333/76; C07D 409/04; H01L 51/0061

(57) ABSTRACT

The present disclosure relates to a novel compound and an organic electroluminescent device including the same. When the compound according to the present disclosure is used in an organic layer, preferably a light-emitting layer, in an organic electroluminescent device, it may improve the luminous efficiency, driving voltage, lifetime, etc. of the organic electroluminescent device.

### TECHNICAL FIELD

The present disclosure relates to a novel organic light-emitting compound, which may be used as a material for an organic electroluminescent device, and an organic electroluminescent device including the same.

### BACKGROUND ART

Studies on an organic electroluminescent (EL) device started with the observation of light emission from an organic thin film by Bernanose in 1950s, which led to blue electroluminescence using an anthracene single crystal in 1965. Since then, an organic electroluminescent device having a stacked structure divided into functional layers including a hole layer and a light-emitting layer was proposed by Tang in 1987 (C. W. Tang, App. Phys. Lett. 1986, 48, 183; C. W. Tang and S. A. VanSlyke, App. Phys. Lett. 1987, 51, 913; A. Tsumura, et. al., App. Phys. Lett. 1986, 49, 1210). Since then, progress has been made by introducing each characteristic organic layer into an organic electroluminescent device to make the organic electroluminescent device having high efficiency and a long lifetime, and a specific material that is used in each organic layer has been developed.

When a voltage is applied across two electrodes in an organic electroluminescent device, holes are injected from the anode into organic layers, and electrons are injected from the cathode into the organic layers. When the injected holes and electrons meet each other, excitons are formed, and when the excitons return to the ground state, light is emitted. At this time, materials that are used as the organic layers may be classified according to their function into light-emitting materials, hole injection materials, hole transport materials, electron transport materials, electron injection materials, and the like.

The light-emitting materials may be classified according to the luminous color into blue, green and red light-emitting materials, and may also be classified into yellow and orange light-emitting materials for achieving better natural colors. In addition, to increase color purity and increase luminous efficiency through energy transfer, a host/dopant system may be used as a light-emitting material.

Dopant materials may be divided into: a fluorescent dopant using an organic material; and a phosphorescent dopant using a metal complex compound containing heavy atoms such as Ir and Pt. The development of phosphorescent materials can theoretically improve luminous efficiency up to four times as compared to fluorescent materials. Therefore, many studies have been conducted not only on phosphorescent dopant materials, but also on phosphorescent host materials.

Until now, NPB, BCP, Alq₃ and the like are widely known as hole injection layer, hole transport layer, hole blocking layer and electron transport layer materials, and anthracene derivatives have been reported as light-emitting layer materials. In particular, among the light-emitting layer materials, metal complex compounds containing Ir, such as Firpic, Ir(ppy)₃ and (acac)Ir(btp)₂, which have an advantage in terms of improving efficiency, have been used as blue, green and red phosphorescent dopant materials, and 4,4-dicarbazolyl biphenyl (CBP) has been used as a phosphorescent host material.

[chemical structure]

Although conventional organic layer materials are advantages in terms of light-emitting properties, they have very poor thermal stability due to their low glass transition temperature, and thus are not satisfactory in terms of the lifetime of organic electroluminescent devices. Therefore, there is a need to develop an organic material layer material having excellent performance.

### DISCLOSURE

### Technical Problem

An object of the present disclosure is to provide a novel organic compound, which is applicable to an organic electroluminescent device and is excellent in terms of all hole and electron injection and transport abilities, light-emitting ability, and the like.

Another object of the present disclosure is to provide an organic electroluminescent device, which includes the novel organic compound, and thus exhibits low-voltage driving characteristics and high luminous efficiency and has improved lifetime, the novel organic compound being used as a hole transport layer material and an auxiliary hole transport layer material.

### Technical Solution

Basically, spiroacridine-based structures have excellent electrochemical stability, high glass transition temperature and excellent carrier transport ability. In particular, these structures have excellent hole transport ability, and thus facilitate hole transport to a light-emitting layer, thus increasing luminous efficiency.

Materials represented by Formula 1 according to the present disclosure are structurally characterized by having spirodimethylfluorene, a spiroacridine core, and arylamine.

In addition, linkers that are selected in the present disclosure have low-voltage driving and high refractive index characteristics based on excellent hole transport ability, and thus exhibit high efficiency and physical properties such as long lifetime.

For this reason, a compound represented by Formula 1 according to the present disclosure has excellent luminescent properties, and thus may be may be used as a material for any one of a hole injection layer, a hole transport layer, a light-emitting layer, an electron transport layer and an electron injection layer, which are the organic layers of an organic electroluminescent device. Preferably, the compound may be used as a hole transport layer material and an auxiliary hole transport layer material.

The present disclosure provides a compound represented by the following Formula 1:

[chemical structure]

wherein

X is N, O, S or C;

L is a direct bond or is selected from the group consisting of C₆-C₁₈ arylene group and a heteroarylene group having 5 to 18 nuclear atoms;

Ar₁ and Ar₂ are each independently selected from the group consisting of hydrogen, deuterium, a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylamine group, and are symmetrical or asymmetrical with respect to each other; and

the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of Ar₁ and Ar₂ are each independently unsubstituted or substituted with one or more substituents selected from the group consisting of deuterium, a halogen, a cyano group, a nitro group, a C₁-C₄₀ alkyl group, a C₂-C₄₀ alkenyl group, a C₂-C₄₀ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents are the same or different.

The present disclosure also provides a compound represented by the following Formula 2:

[chemical structure]

wherein

X, L, Ar₁ and Ar₂ are as defined in Formula 1 above;

R₁ and R₂ are each independently selected from the group consisting of hydrogen, deuterium, halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₃-C₄ alkylsilyl group, a C₆-C₆₀ arylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and C₆-C₆₀ arylamine group; and

the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of R₁ and R₂ are each independently unsubstituted or substituted with one or more substituents selected from the group consisting of deuterium, a halogen, a cyano group, a nitro group, a C₁-C₄₀ alkyl group, a C₂-C₄₀ alkenyl group, a C₂-C₄₀ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents are the same or different.

The present disclosure also provides an organic electroluminescent device including an anode, a cathode, and one or more organic layers interposed between the anode and the cathode, wherein at least one of the one or more organic layers includes the compound of Formula 1 or Formula 2.

“Halogen” in the present disclosure means fluorine, chlorine, bromine or iodine.

“Alkyl” in the present disclosure means a monovalent substituent derived from a C₁-C₄₀ straight or branched-chain saturated hydrocarbon. Examples thereof include, but are not limited to, methyl, ethyl, propyl, isobutyl, sec-butyl, pentyl, iso-amyl, hexyl, and the like.

“Alkenyl” in the present disclosure means a monovalent substituent derived from a C₂-C₄₀ straight or branched-chain unsaturated hydrocarbon having one or more carbon-carbon double bonds. Examples thereof include, but are not limited to, vinyl, allyl, isopropenyl, 2-butenyl, and the like.

“Alkynyl” in the present disclosure means a monovalent substituent derived from a C₂-C₄₀ straight or branched-chain unsaturated hydrocarbon having one or more carbon-carbon triple bonds. Examples thereof include, but are not limited to, ethynyl, 2-propynyl, and the like.

“Aryl” in the present disclosure means a monovalent substituent derived from a C₆-C₆₀ aromatic hydrocarbon having a single ring or a combination of two or more rings. In addition, aryl may also include a monovalent substituent, which has two or more rings fused together, contains only carbon atoms (e.g., 8 to 60 carbon atoms) as ring-forming atoms and has non-aromaticity in the entire molecular structure. Examples of this aryl include, but are not limited to, phenyl, naphthyl, phenanthryl, anthryl, fluorenyl, dimethylfluorenyl, and the like.

“Heteroaryl” in the present disclosure means a monovalent substituent derived from a monoheterocyclic or polyheterocyclic aromatic hydrocarbon having 5 to 60 nuclear atoms. Here, one or more carbon atoms, preferably 1 to 3 carbon atoms, in the ring, are substituted with a heteroatom selected from among N, O, P, S and Se. In addition, heteroaryl is also intended to include a monovalent group, which has two or more rings pendant to each other or fused to each other, contains a heteroatom selected from among N, O, P, S and Se in addition to carbon atoms as ring-forming atoms, and has non-aromaticity in the entire molecular structure. Examples of this heteroaryl include, but are not limited to, 6-membered monocyclic rings such as pyridyl, pyrazinyl, pyrimidinyl, pyridazinyl, and triazinyl; polycyclic rings such as phenoxathienyl, indolizinyl, indolyl, purinyl, quinolyl, benzothiazole, and carbazolyl; 2-furanyl, N-imidazolyl, 2-isoxazolyl, 2-pyridinyl, 2-pyrimidinyl, and the like.

“Aryloxy” in the present disclosure means a monovalent substituent represented by RO—, wherein R represents a C₅ to C₆₀ aryl. Examples of this aryloxy include, but are not limited to, phenyloxy, naphthyloxy, diphenyloxy, and the like.

“Alkyloxy” in the present disclosure means a monovalent substituent represented by R′O—, wherein R′ represents a C₁-C₄₀ alkyl, and is intended to include a linear, branched or cyclic structure. Examples of this alkyloxy include, but are not limited to, methoxy, ethoxy, n-propoxy, 1-propoxy, t-butoxy, n-butoxy, pentoxy, and the like.

“Arylamino” in the present disclosure means an amine substituted with C₆-C₆₀ aryl.

“Cycloalkyl” in the present disclosure means a monovalent substituent derived from a C₃-C₄₀ monocyclic or polycyclic non-aromatic hydrocarbon. Examples of this cycloalkyl include, but are not limited to, cyclopropyl, cyclopentyl, cyclohexyl, norbornyl, adamantine, and the like.

“Heterocycloalkyl” in the present disclosure means a monovalent substituent derived from a non-aromatic hydrocarbon having 3 to 40 nuclear atoms, and one or more carbon atoms, preferably 1 to 3 carbon atoms, in the ring, are substituted with a heteroatom such as N, O, S or Se. Examples of this heterocycloalkyl include, but are not limited to, morpholine, piperazine, and the like.

“Alkylsilyl” in the present disclosure means a silyl substituted with C₁-C₄₀ alkyl, and “arylsilyl” means a silyl substituted with C₅-C₆₀ aryl.

“Fused ring” in the present disclosure means a fused aliphatic ring, a fused aromatic ring, a fused heteroaliphatic ring, a fused heteroaromatic ring, or a combination thereof.

### Advantageous Effects

The compound of the present disclosure is based on a spirodimethylacridine structure, and is characterized by exhibiting excellent luminous efficiency by showing high hole mobility due to excellent hole transport ability.

In addition, the compound of the present disclosure has thermal stability due to high glass transition temperature, and exhibits appropriate HOMO and LUMO energy levels between a hole injection layer and a light-emitting layer, thus enabling low-voltage driving, resulting in an increase in lifetime. Furthermore, since the amorphous properties and high refractive index properties of the compound have the effect of further increasing luminous efficiency, the compound of the present disclosure may be effectively applied to a hole transport layer material and an auxiliary hole transport layer material in an organic electroluminescent device including organic layers.

### MODE FOR INVENTION

Hereinafter, the present disclosure will be described in detail.

1. Novel Organic Compound

The novel compound of the present disclosure may be represented by the following Formula 1:

[chemical structure]

wherein

X is N, O, S or C;

L is a direct bond or is selected from the group consisting of C-Cis arylene group and a heteroarylene group having 5 to 18 nuclear atoms;

Ar₁ and Ar₂ are each independently selected from the group consisting of hydrogen, deuterium, a halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₃-C₄₀ alkylsilyl group, a C₆-C₆₀ arylsilyl group, a C₂-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylamine group, and are symmetrical or asymmetrical with respect to each other; and

the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of Ar₁ and Ar₂ are each independently unsubstituted or substituted with one or more substituents selected from the group consisting of deuterium, a halogen, a cyano group, a nitro group, a C₁-C₄₀ alkyl group, a C₂-C₄₀ alkenyl group, a C₂-C₄₀ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents may be the same or different.

In addition, the novel compound of the present disclosure may be represented by the following Formula 2:

[chemical structure]

wherein

X, L, Ar₁ and Ar₂ are as defined in Formula 1 above;

R₁ and R₂ are each independently selected from the group consisting of hydrogen, deuterium, halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₃-C₄ alkylsilyl group, a C₆-C₆₀ arylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and C₆-C₆₀ arylamine group; and

the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of R₁ and R₂ are each independently unsubstituted or substituted with one or more substituents selected from the group consisting of deuterium, a halogen, a cyano group, a nitro group, a C₁-C₄₀ alkyl group, a C₂-C₄₀ alkenyl group, a C₂-C₄₀ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents may be the same or different.

In one embodiment of the present disclosure, R₁ and R₂ are each independently selected from the group consisting of a C₁-C₄₀ alkyl group, a C₆-C₆₀ aryl group and a heteroaryl group having 5 to 60 nuclear atoms. The alkyl group, aryl group and heteroaryl group of R₁ and R₂ are each independently unsubstituted or substituted with one or more substituents selected from the group consisting of a C₁ to C₄₀ alkyl group, a C₆ to C₆₀ aryl group and a heteroaryl group having 5 to 60 nuclear atoms, and when they are substituted with a plurality of substituents, these substituents may be the same or different.

In a preferred embodiment of the present disclosure, R₁ and R₂ are each independently selected from the group consisting of a methyl group, an ethyl group, a propyl group, a butyl group, a pentyl group, a phenyl group, a biphenyl group, a pyridinyl group, a pyrimidinyl group and a triazinyl group. The methyl group, ethyl group, propyl group, butyl group, pentyl group, phenyl group, biphenyl group, pyridinyl group, pyrimidinyl group and triazinyl group of R₁ and R₂ are each independently unsubstituted or substituted with one or more substituents selected from the group consisting of a C₁-C₄₀ alkyl group, a C₆-C₆₀ aryl group and a heteroaryl group having 5 to 60 nuclear atoms, and when they are substituted with a plurality of substituents, these substituents may be the same or different.

In one embodiment of the present disclosure, L may be a direct bond or one or more linkers selected from the group consisting of the following Formulas 3 to 8:

[chemical structure]

In one embodiment of the present disclosure, Ar₁ and Ar₂ are selected from the group consisting of a C₁-C₄₀ alkyl group, a C₆-C₆₀ aryl group and a heteroaryl group having 5 to 60 nuclear atoms. The alkyl group, aryl group and heteroaryl group of Ar₁ and Ar₂ are each independently unsubstituted or substituted with one or more substituents selected from the group consisting of a C₁-C₄₀ alkyl group, a C₆-C₆₀ aryl group and a heteroaryl group having 5 to 60 nuclear atoms, and when they are substituted with a plurality of substituents, these substituents may be the same or different.

In a preferred embodiment of the present disclosure, Ar₁ and Ar₂ are selected from the group consisting of a methyl group, an ethyl group, a propyl group, a butyl group, a pentyl group, a phenyl group, a biphenyl group, a fluorenyl group, a carbazolyl group, a dibenzofuranyl group, a dibenzothiophenyl group, a pyridinyl group, a pyrimidinyl group, a triazinyl group, a naphthalenyl group, a triazolopyridinyl group, a quinolinyl group, an isoquinolinyl group, a cinnolinyl group, a quinoxalinyl group and a quinazolinyl group. The methyl group, ethyl group, propyl group, butyl group, pentyl group, phenyl group, biphenyl group, fluorenyl group, carbazolyl group, dibenzofuranyl group, dibenzothiophenyl group, pyridinyl group, pyrimidinyl group, triazinyl group, naphthalenyl group, triazolopyridinyl group, quinolinyl group, isoquinolinyl group, cinnolinyl group, quinoxalinyl group and quinazolinyl group of Ar₁ and Ar₂ are each independently unsubstituted or substituted with one or more substituents selected from the group consisting of a C₁-C₄₀ alkyl group, a C₆-C₆₀ aryl group and a heteroaryl group having 5 to 60 nuclear atoms, and when they are substituted with a plurality of substituents, these substituents may be the same or different.

In one embodiment of the present disclosure, the compound represented by Formula 1 or Formula 2 may be a compound represented by the following formula, which is 7-(10,10-dimethyl-10H-spiro[anthracene-9,9′-fluoren]-3′-yl)-N,N-diphenyldibenzo[b,d]furan-2-amine or N,N-di([1,1′-biphenyl]-4-yl)-7-(10-phenyl-10H-spiro[acridine-9,9′-fluoren]-2′-yl)phenanthren-2-amine:

[chemical structure]

In one embodiment of the present disclosure, the compound represented by Formula 1 or Formula 2 may be selected from the group consisting of the following compounds, but is not limited thereto:

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

The compound of Formula 1 according to the present disclosure may be synthesized according to a general synthesis method (see Chem. Rev., 60:313 (1960); J. Chem. SOC. 4482 (1955); Chem. Rev. 95: 2457 (1995), etc.). A detailed synthesis process for the compound of the present disclosure will be described in detail in the Synthesis Examples below.

2. Organic Electroluminescent Device

The present disclosure is also directed to an organic electroluminescent (EL) device including the compound represented by Formula 1 or Formula 2 according to the present disclosure.

Specifically, the present disclosure is directed to an organic electroluminescent device including an anode, a cathode, and one or more organic layers interposed between the anode and the cathode, wherein at least one of the one or more organic layers includes the compound represented by Formula 1 or Formula 2. Here, the compound may be used alone or as a mixture of two or more.

The one or more organic layers may be any one or more of a hole injection layer, a hole transport layer, an auxiliary hole transport layer, an electron injection layer, an electron transport layer, an auxiliary electron transport layer, and a light-emitting layer, and at least one organic layer among these layers may include the compound represented by Formula 1.

The structure of the organic electroluminescent device according to the present disclosure is not particularly limited, and may include, for example, an anode and a cathode, which are opposite to each other, and organic layers interposed between the anode and the cathode. Here, the organic layers may include a hole transport layer, a light-emitting layer and an electron transport layer. In addition, the organic layers may include an auxiliary hole transport layer between the hole transport layer and the light-emitting layer, and may include an auxiliary electron transport layer between the electron transport layer and the light-emitting layer.

In addition, the organic layers may further include a hole injection layer between the hole transport layer and the anode, and may further include an electron injection layer between the electron transport layer and the cathode.

In the present disclosure, the hole injection layer disposed between the hole transport layer and the anode is a layer functioning not only to improve the interfacial characteristics between the ITO used as the anode and an organic material used as the hole transport layer, but also to smooth the surface of ITO by being applied on the ITO whose surface is not flat. For the hole injection layer, any material may be used without particular limitation as long as it is commonly used in the art. For example, an amine compound may be used, but the present disclosure is not limited thereto.

In addition, the electron injection layer is a layer which is disposed on the electron transport layer and functions to facilitate electron injection from the cathode, thus ultimately improving power efficiency. For the electron injection layer, any material may be used without particular limitation as long as it is commonly used in the art. For example, a material such as LiF, Liq, NaCl, CsF, Li₂O or BaO may be used.

In addition, the organic layers may further include an auxiliary light-emitting layer between the auxiliary hole transport layer and the light-emitting layer. The auxiliary light-emitting layer may function to adjust the thicknesses of the organic layers while transporting holes to the light-emitting layer. The auxiliary light-emitting layer may include a hole transport material and may be made of the same material as the hole transport layer.

In addition, the organic layers may further include a lifetime improvement layer between the auxiliary electron transport layer and the light-emitting layer. Holes moving depending on ionization potential levels to the light-emitting layer in the organic electroluminescent device are blocked by the high energy barrier of the lifetime improvement layer, and thus cannot diffuse or move to the electron transport layer. As a result, the lifetime improvement layer functions to confine holes to the light-emitting layer. This function of confining holes to the light-emitting layer may prevent holes from diffusing to the electron transport layer that moves electrons by reduction, suppressing the lifetime from decreasing due to irreversible decomposition reactions caused by oxidation, thus contributing to improvement in the lifetime of the organic electroluminescent device.

Basically, spiroacridine-based structures have excellent electrochemical stability, high glass transition temperature and excellent carrier transport ability. In particular, these structures have excellent hole transport ability, and thus facilitate hole transport to a light-emitting layer, thus increasing luminous efficiency.

The compound represented by Formula 1 according to the present disclosure is structurally characterized by having spirodimethylfluorene and arylamine, has low-voltage driving and high refractive index characteristics, and thus exhibits high efficiency and physical properties such as long lifetime.

For this reason, the compound represented by Formula 1, which is the representative claimed structure of the present disclosure, has excellent luminescent properties, and thus may be used as a material for any one of a hole injection layer, a hole transport layer, an auxiliary hole transport layer, an electron injection layer, an electron transport layer, an auxiliary electron transport layer and a light-emitting layer, which are the organic layers of an organic electroluminescent device. Preferably, the compound may be used as a hole transport layer material and an auxiliary hole transport layer material.

In addition, in the present disclosure, the organic electroluminescent device include an anode, one or more organic layers and a cathode which are sequentially stacked, and may also further include an insulating layer or an adhesive layer at the interface between the electrode and the organic layer.

The organic electroluminescent device of the present disclosure may be fabricated by forming other organic layers and electrodes using the materials and methods known in the related art, except that at least one (e.g., an auxiliary electron transport layer) of the organic layers is formed to include the compound represented by Formula 1.

The organic material layers may be formed by a vacuum vapor deposition method or a solution application method. Examples of the solution application method include, but are not limited to, spin coating, dip coating, doctor blading, inkjet printing, and thermal transfer.

Substrates that may be used in the present disclosure are not particularly limited, and include silicon wafers, quartz, glass sheets, metal sheets, plastic films and sheets, etc.

In addition, as an anode material, for example, a conductive material having a high work function may be used so that hole injection can be facilitated. Examples of the anode material include, but are not limited to, metals such as vanadium, chromium, copper, zinc and gold, or alloys thereof; metal oxides such as zinc oxide, indium oxide, indium tin oxide (ITO), and indium zinc oxide (IZO); a metal/oxide combination such as ZnO:Al or SnO₂:Sb; conductive polymers such as polythiophene, poly(3-methylthiophene), poly(3,4-(ethylene-1,2-dioxy)thiophene (PEDT), polypyrrole or polyaniline; and carbon black.

In addition, as a cathode material, for example, a conductive material having a low work function may be used so that electron injection can be facilitated. Examples of the cathode material include, but are not limited to, metals such as magnesium, calcium, sodium, potassium, titanium, indium, yttrium, lithium, gadolinium, aluminum, silver, tin or lead, or alloys thereof; and multilayer structure materials such as LiF/Al or LiO₂/Al.

Hereinafter, the present disclosure will be described in detail with reference to Examples. However, the following Examples are only to illustrate the present disclosure, and the scope of the present disclosure is not limited by these Examples.

### EXAMPLES

### [Preparation Example 1] Synthesis of 2′-(4-chloronaphthalen-1-yl)-10,10-dimethyl-10H-spiro[anthracene-9,9′-fluorene]

[chemical structure]

200 mL of toluene, 50 mL of EtOH and 50 mL of H₂O were added to 15.0 g (38.2 mmol) of 2′-chloro-10,10-dimethyl-10H-spiro[anthracene-9,9′-fluorene] and 9.5 g (45.9 mmol) of (4-chloronaphthalen-1-yl)boronic acid. 2.3 g (2.0 mmol) of Pd(PPh₃)₄ and 10.6 g (76.4 mmol) of K₂CO₃ were added thereto, followed by heating under reflux at 100° C. for 3 hours. The reaction was terminated by lowering the temperature to room temperature and adding 300 mL of purified water to the reaction solution. The reaction mixture was extracted with 1.0 L of ethyl acetate (EA) and then washed with distilled water. The obtained organic layer was dried with anhydrous MgSO₄, distilled under reduced pressure, and purified by silica gel column chromatography to obtain 12.3 g (62% yield) of the desired compound.

[LCMS]: 519

### [Preparation Example 2] Synthesis of 3′-(4-chloronaphthalen-1-yl)-10,10-dimethyl-10H-spiro[anthracene-9,9′-fluorene]

[chemical structure]

19.5 g (49%) of the desired compound was obtained by performing the same process as Preparation Example 1, except that 3′-chloro-10,10-dimethyl-10H-spiro[anthracene-9,9′-fluorene] was used as the reactant.

[LCMS]: 519

### [Preparation Example 3] Synthesis of 4′-(4-chloronaphthalen-1-yl)-10,10-dimethyl-10H-spiro[anthracene-9,9′-fluorene]

[chemical structure]

20.4 g (69%) of the desired compound was obtained by performing the same process as Preparation Example 1, except that 4′-bromo-10,10-dimethyl-10H-spiro[anthracene-9,9′-fluorene] was used as the reactant.

[LCMS]: 519

### [Preparation Example 4] Synthesis of 2′-(4-chloronaphthalen-1-yl)-10,10-diphenyl-10H-spiro[anthracene-9,9′-fluorene]

[chemical structure]

18.6 g (55%) of the desired compound was obtained by performing the same process as Preparation Example 1, except that 2′-chloro-10,10-diphenyl-10H-spiro[anthracene-9,9′-fluorene] was used as the reactant.

[LCMS]: 643

### [Preparation Example 5] Synthesis of 2′-(4-chloronaphthalen-1-yl)-10-phenyl-10H-spiro[acridine-9,9′-fluorene]

[chemical structure]

21.5 g (68%) of the desired compound was obtained by performing the same process as Preparation Example 1, except that 2′-bromo-10-phenyl-10H-spiro[acridine-9,9′-fluorene] was used as the reactant.

[LCMS]: 568

### [Preparation Example 6] Synthesis of 3′-(4-chloronaphthalen-1-yl)-10-phenyl-10H-spiro[acridine-9,9′-fluorene]

[chemical structure]

18.0 g (61%) of the desired compound was obtained by performing the same process as Preparation Example 1, except that 3′-bromo-10-phenyl-10H-spiro[acridine-9,9′-fluorene] was used as the reactant.

[LCMS]: 568

### [Preparation Example 7] Synthesis of 3′-(4-chloronaphthalen-1-yl)-10-phenyl-10H-spiro[acridine-9,9′-fluorene]

[chemical structure]

11.3 g (42%) of the desired compound was obtained by performing the same process as Preparation Example 1, except that 4′-chloro-10-phenyl-10H-spiro[acridine-9,9′-fluorene] was used as the reactant.

[LCMS]: 568

### [Preparation Example 8] Synthesis of 2′-(6-chloronaphthalen-2-yl)-10,10-dimethyl-10H-spiro[anthracene-9,9′-fluorene]

[chemical structure]

15.5 g (39%) of the desired compound was obtained by performing the same process as Preparation Example 1, except that 4′-chloro-10-phenyl-10H-spiro[acridine-9,9′-fluorene] and (6-chloronaphthalen-2-yl)boronic acid were used as the reactants.

[LCMS]: 519.

### [Preparation Example 9] Synthesis of 4′-(6-chloronaphthalen-2-yl)-10,10-dimethyl-10H-spiro[anthracene-9,9′-fluorene]

[chemical structure]

23.4 g (62%) of the desired compound was obtained by performing the same process as Preparation Example 8, except that 4′-bromo-10,10-dimethyl-10H-spiro[anthracene-9,9′-fluorene] was used as the reactant.

[LCMS]: 519.

### [Preparation Example 10] Synthesis of 3′-(6-chloronaphthalen-2-yl)-10-phenyl-10H-spiro[acridine-9,9′-fluorene]

[chemical structure]

28.0 g (75%) of the desired compound was obtained by performing the same process as Preparation Example 8, except that 3′-bromo-10-phenyl-10H-spiro[acridine-9,9′-fluorene] was used as the reactant.

[LCMS]: 568

### [Preparation Example 11] Synthesis of 2′-(7-chlorophenanthren-2-yl)-10,10-dimethyl-10H-spiro[anthracene-9,9′-fluorene]

[chemical structure]

13.3 g (44%) of the desired compound was obtained by performing the same process as Preparation Example 1, except that 2′-chloro-10,10-dimethyl-10H-spiro[anthracene-9,9′-fluorene] and (7-chlorophenanthren-2-yl)boron acid were used as the reactants.

[LCMS]: 569

### [Preparation Example 12] Synthesis of 2′-(7-chlorophenanthren-2-yl)-10,10-dimethyl-10H-spiro[anthracene-9,9′-fluorene]

[chemical structure]

17.5 g (70%) of the desired compound was obtained by performing the same process as Preparation Example 11, except that 4′-bromo-10,10-dimethyl-10H-spiro[anthracene-9,9′-fluorene] was used as the reactant.

[LCMS]: 569

### [Preparation Example 13] Synthesis of 4′-(7-chlorophenanthren-2-yl)-10,10-diphenyl-10H-spiro[anthracene-9,9′-fluorene]

[chemical structure]

20.2 g (75%) of the desired compound was obtained by performing the same process as Preparation Example 11, except that 4′-bromo-10,10-diphenyl-10H-spiro[anthracene-9,9′-fluorene] was used as the reactant.

[LCMS]: 693

### [Preparation Example 14] Synthesis of 2′-(7-chlorophenanthren-2-yl)-10-phenyl-10H-spiro[acridine-9,9′-fluorene]

[chemical structure]

14.4 g (68%) of the desired compound was obtained by performing the same process as Preparation Example 11, except that 2′-bromo-10-phenyl-OH-spiro[acridine-9,9′-fluorene] was used as the reactant.

[LCMS]: 618

### [Preparation Example 15] Synthesis of 4-bromo-6-(10,10-dimethyl-10H-spiro[anthracene-9,9′-fluoren]-2′-yl)dibenzo[b,d]furan

[chemical structure]

10.5 g (59%) of the desired compound was obtained by performing the same process as Preparation Example 1, except that 2-(10,10-dimethyl-OH-spiro[anthracene-9,9′-fluoren]-2′-yl)-4,4,5,5-tetramethyl-1,3,2-dioxaborolan and 4,6-dibromobenzo[b,d]furan were used as the reactants.

[LCMS]: 603

### [Preparation Example 16] Synthesis of 4-bromo-6-(10,10-dimethyl-10H-spiro[anthracene-9,9′-fluoren]-4′-yl)dibenzo[b,d]furan

[chemical structure]

12.5 g (51%) of the desired compound was obtained by performing the same process as Preparation Example 15, except that 2-(10,10-dimethyl-10H-spiro[anthracene-9,9′-fluoren]-4′-yl)-4,4,5,5-tetramethyl-1,3,2-dioxaborolane was used as the reactant.

[LCMS]: 603

### [Preparation Example 17] Synthesis of 2-chloro-8-(10,10-dimethyl-10H-spiro[anthracene-9,9′-fluoren]-2′-yl)dibenzo[b,d]furan

[chemical structure]

20.2 g (62%) of the desired compound was obtained by performing the same process as Preparation Example 1, except that 2-(10,10-dimethyl-OH-spiro[anthracene-9,9′-fluoren]-2′-yl)-4,4,5,5-tetramethyl-1,3,2-dioxaborolane and 2-bromo-8-chlorodibenzo[b,d]furan were used as the reactants.

[LCMS]: 559

### [Preparation Example 18] Synthesis of 4′-(6-bromodibenzo[b,d]furan-4-yl)-10-phenyl-10H-spiro[acridine-9,9′-fluorene]

[chemical structure]

9.7 g (38%) of the desired compound was obtained by performing the same process as Preparation Example 15, except that 10-phenyl-4′-(4,4,5,5-tetramethyl-1,3,2-dioxaborolan-2-yl)-10H-spiro[acridine-9,9′-fluorene] and 4,6-dibromodibenzo[b,d]furan were used as the reactants.

[LCMS]: 652

### [Preparation Example 19] Synthesis of 4′-(8-chlorodibenzo[b,d]furan-2-VI)-10-phenyl-10H-spiro[acridine-9,9′-fluorene]

[chemical structure]

14.4 g (68%) of the desired compound was obtained by performing the same process as Preparation Example 1, except that 10-phenyl-4′-(4,4,5,5-tetramethyl-1,3,2-dioxaborolan-2-yl)-10H-spiro[acridine-9,9′-fluorene] and 2-bromo-8-chlorodibenzo[b,d]furan were used as the reactants.

[LCMS]: 608

### [Preparation Example 20] Synthesis of 4-bromo-6-(10,10-dimethyl-10H-spiro[anthracene-9,9′-fluoren]-2′-yl)dibenzo[b,d]thiophene

[chemical structure]

8.5 g (42%) of the desired compound was obtained by performing the same process as Preparation Example 1, except that 2-(10,10-dimethyl-OH-spiro[anthracene-9,9′-fluoren]-2′-yl)-4,4,5,5-tetramethyl-1,3,2-dioxaborolane and 4,6-dibromodibenzo[b,d]thiophene were used as the reactants.

[LCMS]: 619

### [Preparation Example 21] Synthesis of 2-chloro-8-(10,10-dimethyl-10H-spiro[anthracene-9,9′-fluoren]-4′-yl)dibenzo[b,d]thiophene

[chemical structure]

19.0 g (66%) of the desired compound was obtained by performing the same process as Preparation Example 1, except that 2-(10,10-dimethyl-OH-spiro[anthracene-9,9′-fluoren]-4′-yl)-4,4,5,5-tetramethyl-1,3,2-dioxaborolane and 2-bromo-8-chlorodibenzo[b,d]thiophene were used as the reactants.

[LCMS]: 575

### [Preparation Example 22] Synthesis of 2′-(8-chlorodibenzo[b,d]thiophen-2-yl)-10-phenyl-10H-spiro[acridine-9,9′-fluorene]

[chemical structure]

12.5 g (60%) of the desired compound was obtained by performing the same process as Preparation Example 1, except that 10-phenyl-2′-(4,4,5,5-tetramethyl-1,3,2-dioxaborolan-2-yl)-10H-spiro[acridine-9,9′-fluorene] and 2-bromo-8-chlorobenzo[b,d]thiophene were used as the reactants.

[LCMS]: 624

### [Preparation Example 23] Synthesis of 3′-(7-bromo-9,9-dimethyl-9H-fluoren-2-yl)-10,10-dimethyl-10H-spiro[anthracene-9,9′-fluorene]

[chemical structure]

8.8 g (45%) of the desired compound was obtained by performing the same process as Preparation Example 1, except that 2-(10,10-dimethyl-OH-spiro[anthracene-9,9′-fluoren]-3′-yl)-4,4,5,5-tetramethyl-1,3,2-dioxaborolane and 2,7-dibromo-9,9-dimethyl-9H-fluorene were used as the reactants.

[LCMS]: 629

### [Preparation Example 24] Synthesis of 2′-(7-bromo-9,9-dimethyl-9H-fluoren-2-yl)-10,10-diphenyl-10H-spiro[anthracene-9,9′-fluorene]

[chemical structure]

4.1 g (39%) of the desired compound was obtained by performing the same process as Preparation Example 1, except that 2-(10,10-diphenyl-OH-spiro[anthracene-9,9′-fluoren]-2′-yl)-4,4,5,5-tetramethyl-1,3,2-dioxaborolane and 2,7-dibromo-9,9-dimethyl-9H-fluorene were used as the reactants.

[LCMS]: 753

### [Preparation Example 25] Synthesis of 4′-(7-bromo-9,9-dimethyl-9H-fluoren-2-yl)-10-phenyl-10H-spiro[acridine-9,9′-fluorene]

[chemical structure]

5.0 g (46%) of the desired compound was obtained by performing the same process as Preparation Example 1, except that 10-phenyl-4′-(4,4,5,5-tetramethyl-1,3,2-dioxaborolan-2-yl)-10H-spiro[acridine-9,9′-fluorene] and 2,7-dibromo-9,9-dimethyl-9H-fluorene were used as the reactants.

[LCMS]: 678

### [Synthesis Example 1] Synthesis of Compound 1

[chemical structure]

To 10.2 g (19.6 mmol) of the compound of Preparation Example 1 and 3.0 g (17.8 mmol) of diphenylamine, 100 mL of toluene was added. 0.82 g (0.9 mmol) of Pd₂(dba)₃, 0.86 g (1.8 mmol) of XPhos and 2.6 g (26.7 mmol) of NaOt-Bu were added to the reaction solution which was then heated under reflux at 120° C. for 5 hours. The reaction was terminated by lowering the temperature to room temperature and adding 300 mL of purified water to the reaction solution. The mixture was extracted with 500 mL of ethyl acetate (EA) and then washed with distilled water. The obtained organic layer was dried with anhydrous MgSO₄, distilled under reduced pressure, and purified by silica gel column chromatography to obtain 8.5 g (73% yield) of the desired compound.

[LCMS]: 651

### [Synthesis Example 2] Synthesis of Compound 2

[chemical structure]

10.5 g (80% yield) of the desired compound was obtained by performing the same process as Synthesis Example 1, except that 2′-(4-chloronaphthalen-1-yl)-10,10-dimethyl-10H-spiro[anthracene-9,9′-fluorene of Preparation Example 1 and N-phenyl-[1,1′-biphenyl]-4-amine were used.

[LCMS]: 727

### [Synthesis Example 3] Synthesis of Compound 8

[chemical structure]

7.7 g (52% yield) of the desired compound was obtained by performing the same process as Synthesis Example 1, except that 3′-(4-chloronaphthalen-1-yl)-10,10-dimethyl-10H-spiro[anthracene-9,9′-fluorene] of Preparation Example 2 and di([1,1′-biphenyl]-4-yl)amine were used.

[LCMS]: 804

### [Synthesis Example 4] Synthesis of Compound 9

[chemical structure]

7.7 g (52% yield) of the desired compound was obtained by performing the same process as Synthesis Example 1, except that 3′-(4-chloronaphthalen-1-yl)-10,10-dimethyl-10H-spiro[anthracene-9,9′-fluorene] of Preparation Example 2 and N-([1,1′-biphenyl]-4-yl)-[1,1′-biphenyl]-2-amine were used.

[LCMS]: 804

### [Synthesis Example 5] Synthesis of Compound 12

[chemical structure]

3.8 g (72% yield) of the desired compound was obtained by performing the same process as Synthesis Example 1, except that 4′-(4-chloronaphthalen-1-yl)-10,10-dimethyl-10H-spiro[anthracene-9,9′-fluorene] of Preparation Example 3 and N-phenyl-[1,1′-biphenyl]-4-amine were used.

[LCMS]: 727

### [Synthesis Example 6] Synthesis of Compound 15

[chemical structure]

5.0 g (60% yield) of the desired compound was obtained by performing the same process as Synthesis Example 1, except that 4′-(4-chloronaphthalen-1-yl)-10,10-dimethyl-10H-spiro[anthracene-9,9′-fluorene] of Preparation Example 3 and N-([1,1′-biphenyl]-4-yl)-9,9-dimethyl-9H-fluorene-2-amine were used.

[LCMS]: 844

### [Synthesis Example 7] Synthesis of Compound 16

[chemical structure]

6.3 g (75% yield) of the desired compound was obtained by performing the same process as Synthesis Example 1, except that 2′-(4-chloronaphthalen-1-yl)-10,10-diphenyl-10H-spiro[anthracene-9,9′-fluorene] of Preparation Example 4 and diphenylamine were used.

[LCMS]: 776

### [Synthesis Example 8] Synthesis of Compound 22

[chemical structure]

4.4 g (69% yield) of the desired compound was obtained by performing the same process as Synthesis Example 1, except that 2′-(4-chloronaphthalen-1-yl)-10-phenyl-10H-spiro[acridine-9,9′-fluorene] of Preparation Example 5 and N-phenyl-[1,1′-biphenyl]-4-amine were used.

[LCMS]: 776

### [Synthesis Example 9] Synthesis of Compound 24

[chemical structure]

8.4 g (59% yield) of the desired compound was obtained by performing the same process as Synthesis Example 1, except that 2′-(4-chloronaphthalen-1-yl)-10-phenyl-10H-spiro[acridine-9,9′-fluorene] of Preparation Example 5 and N-([1,1′-biphenyl]-4-yl)-[1,1′-biphenyl]-2-amine were used.

[LCMS]: 853

### [Synthesis Example 10] Synthesis of Compound 28

[chemical structure]

5.2 g (69% yield) of the desired compound was obtained by performing the same process as Synthesis Example 1, except that 3′-(4-chloronaphthalen-1-yl)-10-phenyl-10H-spiro[acridine-9,9′-fluorene] of Preparation Example 6 and di([1,1′-biphenyl]-4-yl)amine were used.

[LCMS]: 853

### [Synthesis Example 11] Synthesis of Compound 31

[chemical structure]

3.3 g (59% yield) of the desired compound was obtained by performing the same process as Synthesis Example 1, except that 4′-(4-chloronaohthalen-1-yl)-10-phenyl-10H-spiro[acridine-9,9′-fluorene] of Preparation Example 7 and diphenylamine were used.

[LCMS]: 700

### [Synthesis Example 12] Synthesis of Compound 35

[chemical structure]

5.1 g (62% yield) of the desired compound was obtained by performing the same process as Synthesis Example 1, except that 4′-(4-chloronaohthalen-1-yl)-10-phenyl-10H-spiro[acridine-9,9′-fluorene] of Preparation Example 7 and N-([1,1′-biphenyl]-4-yl)-9,9-dimethyl-9H-fluorene-2-amine were used.

[LCMS]: 893

### [Synthesis Example 13] Synthesis of Compound 37

[chemical structure]

5.5 g (70% yield) of the desired compound was obtained by performing the same process as Synthesis Example 1, except that 2′-(6-chloronaphthalen-2-yl)-10,10-dimethyl-10H-spiro[anthracene-9,9′-fluorene] of Preparation Example 8 and N-phenyl-[1,1′-biphenyl]-4-amine were used.

[LCMS]: 727

### [Synthesis Example 14] Synthesis of Compound 39

[chemical structure]

3.9 g (65% yield) of the desired compound was obtained by performing the same process as Synthesis Example 1, except that 2′-(6-chloronaphthalen-2-yl)-10,10-dimethyl-10H-spiro[anthracene-9,9′-fluorene] of Preparation Example 8 and N-([1,1′-biphenyl]-4-yl)-[1,1′-biphenyl]-2-amine were used.

[LCMS]: 804

### [Synthesis Example 15] Synthesis of Compound 48

[chemical structure]

5.1 g (49% yield) of the desired compound was obtained by performing the same process as Synthesis Example 1, except that 4′-(6-chloronaphthalen-2-yl)-10,10-dimethyl-10H-spiro[anthracene-9,9′-fluorene] of Preparation Example 9 and di([1,1′-biphenyl]-4-yl)amine were used.

[LCMS]: 804

### [Synthesis Example 16] Synthesis of Compound 50

[chemical structure]

3.3 g (65% yield) of the desired compound was obtained by performing the same process as Synthesis Example 1, except that 4′-(6-chloronaphthalen-2-yl)-10,10-dimethyl-10H-spiro[anthracene-9,9′-fluorene] of Preparation Example 9 and N-([1,1′-biphenyl]-4-yl)-9,9-dimethyl-9H-fluorene-2-amine were used.

[LCMS]: 844

### [Synthesis Example 17] Synthesis of Compound 57

[chemical structure]

4.0 g (62% yield) of the desired compound was obtained by performing the same process as Synthesis Example 1, except that 3′-(6-chloronaphthalen-2-yl)-10-phenyl-10H-spiro[acridine-9,9′-fluorene] of Preparation Example 10 and N-phenyl-[1,1′-biphenyl]-4-amine were used.

[LCMS]: 776

### [Synthesis Example 18] Synthesis of Compound 58

[chemical structure]

2.9 g (66% yield) of the desired compound was obtained by performing the same process as Synthesis Example 1, except that 3′-(6-chloronaphthalen-2-yl)-10-phenyl-10H-spiro[acridine-9,9′-fluorene] of Preparation Example 10 and di([1,1′-biphenyl]-4-yl)amine were used.

[LCMS]: 853

### [Synthesis Example 19] Synthesis of Compound 66

[chemical structure]

4.1 g (60% yield) of the desired compound was obtained by performing the same process as Synthesis Example 1, except that 2′-(7-chlorophenanthren-2-yl)-10,10-dimethyl-10H-spiro[anthracene-9,9′-fluorene] of Preparation Example 11 and diphenylamine were used.

[LCMS]: 701

### [Synthesis Example 20] Synthesis of Compound 70

[chemical structure]

5.3 g (65% yield) of the desired compound was obtained by performing the same process as Synthesis Example 1, except that 2′-(7-chlorophenanthren-2-yl)-10,10-dimethyl-10H-spiro[anthracene-9,9′-fluorene] of Preparation Example 11 and N-([1,1′-biphenyl]-4-yl)-9,9-dimethyl-9H-fluorene-2-amine were used.

[LCMS]: 894

### [Synthesis Example 21] Synthesis of Compound 77

[chemical structure]

2.5 g (69% yield) of the desired compound was obtained by performing the same process as Synthesis Example 1, except that 4′-(7-chlorophenanthren-2-yl)-10,10-dimethyl-10H-spiro[anthracene-9,9′-fluorene] of Preparation Example 12 and N-phenyl-[1,1′-biphenyl]-4-amine were used.

[LCMS]: 778

### [Synthesis Example 22] Synthesis of Compound 79

[chemical structure]

5.9 g (50% yield) of the desired compound was obtained by performing the same process as Synthesis Example 1, except that 4′-(7-chlorophenanthren-2-yl)-10,10-dimethyl-10H-spiro[anthracene-9,9′-fluorene] of Preparation Example 12 and N-([1,1′-biphenyl]-4-yl)-[1,1′-biphenyl]-2-amine were used.

[LCMS]: 854

### [Synthesis Example 23] Synthesis of Compound 86

[chemical structure]

3.5 g (44% yield) of the desired compound was obtained by performing the same process as Synthesis Example 1, except that 4′-(7-chlorophenanthren-2-yl)-10,10-diphenyl-10H-spiro[anthracene-9,9′-fluorene] of Preparation Example 13 and diphenylamine were used.

[LCMS]: 826

### [Synthesis Example 24] Synthesis of Compound 88

[chemical structure]

4.1 g (39% yield) of the desired compound was obtained by performing the same process as Synthesis Example 1, except that 4′-(7-chlorophenanthren-2-yl)-10,10-diphenyl-10H-spiro[anthracene-9,9′-fluorene] of Preparation Example 13 and di([1,1′-biphenyl]-4-yl)amine were used.

[LCMS]: 978

### [Synthesis Example 25] Synthesis of Compound 94

[chemical structure]

8.5 g (60% yield) of the desired compound was obtained by performing the same process as Synthesis Example 1, except that 2′-(7-chlorophenanthren-2-yl)-10-phenyl-10H-spiro[acridine-9,9′-fluorene] of Preparation Example 14 and N-([1,1′-biphenyl]-4-yl)-[1,1′-biphenyl]-2-amine were used.

[LCMS]: 903

### [Synthesis Example 26] Synthesis of Compound 102

[chemical structure]

6.6 g (49% yield) of the desired compound was obtained by performing the same process as Synthesis Example 1, except that 4-bromo-6-(10,10-dimethyl-OH-spiro[anthracene-9,9′-fluoren]-2′-yl)dibenzo[b,d]furan of Preparation Example 15 and N-phenyl-[1,1′-biphenyl]-4-amine were used.

[LCMS]: 767

### [Synthesis Example 27] Synthesis of Compound 103

[chemical structure]

9.6 g (59% yield) of the desired compound was obtained by performing the same process as Synthesis Example 1, except that 4-bromo-6-(10,10-dimethyl-OH-spiro[anthracene-9,9′-fluoren]-2′-yl)dibenzo[b,d]furan of Preparation Example 15 and di([1,1′-biphenyl]-4-yl)amine were used.

[LCMS]: 844

### [Synthesis Example 28] Synthesis of Compound 109

[chemical structure]

8.4 g (59% yield) of the desired compound was obtained by performing the same process as Synthesis Example 1, except that 4-bromo-6-(10,10-dimethyl-10H-spiro[anthracene-9,9′-fluoren]-4′-yl)dibenzo[b,d]furan of Preparation Example 16 and N-([1,1′-biphenyl]-4-yl)-[1,1′-biphenyl]-2-amine were used.

[LCMS]: 844

### [Synthesis Example 29] Synthesis of Compound 110

[chemical structure]

5.6 g (68% yield) of the desired compound was obtained by performing the same process as Synthesis Example 1, except that 4-bromo-6-(10,10-dimethyl-10H-spiro[anthracene-9,9′-fluoren]-4′-yl)dibenzo[b,d]furan of Preparation Example 16 and N-([1,1′-biphenyl]-4-yl)-9,9-dimethyl-9H-fluorene-2-amine were used.

[LCMS]: 884

### [Synthesis Example 30] Synthesis of Compound 113

[chemical structure]

4.0 g (69% yield) of the desired compound was obtained by performing the same process as Synthesis Example 1, except that 2-chloro-8-(10,10-dimethyl-OH-spiro[anthracene-9,9′-fluoren]-2′-yl)dibenzo[b,d]furan of Preparation Example 17 and di([1,1′-biphenyl]-4-yl)amine were used.

[LCMS]: 884

### [Synthesis Example 31] Synthesis of Compound 126

[chemical structure]

5.1 g (60% yield) of the desired compound was obtained by performing the same process as Synthesis Example 1, except that 4′-(6-bromodibenzo[b,d]furan-4-yl)-10-phenyl-10H-spiro[acridine-9,9′-fluorene] of Preparation Example 18 and diphenylamine were used.

[LCMS]: 740

### [Synthesis Example 32] Synthesis of Compound 129

[chemical structure]

3.0 g (72% yield) of the desired compound was obtained by performing the same process as Synthesis Example 1, except that 4′-(6-bromodibenzo[b,d]furan-4-yl)-10-phenyl-10H-spiro[acridine-9,9′-fluorene] of Preparation Example 18 and N-([1,1′-biphenyl]-4-yl)-[1,1′-biphenyl]-2-amine were used.

[LCMS]: 893

### [Synthesis Example 33] Synthesis of Compound 138

[chemical structure]

5.9 g (60% yield) of the desired compound was obtained by performing the same process as Synthesis Example 1, except that 4′-(8-chlorodibenzo[b,d]furan-2-yl)-10-phenyl-10H-spiro[acridine-9,9′-fluorene] of Preparation Example 19 and di([1,1′-biphenyl]-4-yl)amine were used.

[LCMS]: 893

### [Synthesis Example 34] Synthesis of Compound 142

[chemical structure]

4.5 g (63% yield) of the desired compound was obtained by performing the same process as Synthesis Example 1, except that 4-bromo-6-(10,10-dimethyl-10H-spiro[anthracene-9,9′-fluoren]-2′-yl)dibenzo[b,d]thiophene of Preparation Example 20 and N-phenyl-[1,1′-biphenyl]-4-amine were used.

[LCMS]: 784

### [Synthesis Example 35] Synthesis of Compound 145

[chemical structure]

2.8 g (70% yield) of the desired compound was obtained by performing the same process as Synthesis Example 1, except that 4-bromo-6-(10,10-dimethyl-10H-spiro[anthracene-9,9′-fluoren]-2′-yl)dibenzo[b,d]thiophene of Preparation Example 20 and N-([1,1′-biphenyl]-4-yl)-9,9-dimethyl-9H-fluorene-2-amine were used.

[LCMS]: 900

### [Synthesis Example 36] Synthesis of Compound 156

[chemical structure]

3.5 g (62% yield) of the desired compound was obtained by performing the same process as Synthesis Example 1, except that 2-chloro-8-(10,10-dimethyl-OH-spiro[anthracene-9,9′-fluoren]-4′-yl)dibenzo[b,d]thiophene of Preparation Example 21 and diphenylamine were used.

[LCMS]: 707

### [Synthesis Example 37] Synthesis of Compound 157

[chemical structure]

3.0 g (72% yield) of the desired compound was obtained by performing the same process as Synthesis Example 1, except that 2-chloro-8-(10,10-dimethyl-10H-spiro[anthracene-9,9′-fluoren]-4′-yl)dibenzo[b,d]thiophene of Preparation Example 21 and N-phenyl-[1,1′-biphenyl]-4-amine were used.

[LCMS]: 784

### [Synthesis Example 38] Synthesis of Compound 171

[chemical structure]

6.4 g (62% yield) of the desired compound was obtained by performing the same process as Synthesis Example 1, except that 2′-(8-chlorodibenzo[b,d]thiophen-2-yl)-10-phenyl-10H-spiro[acridine-9,9′-fluorene] of Preparation Example 22 and diphenylamine were used.

[LCMS]: 756

### [Synthesis Example 39] Synthesis of Compound 174

[chemical structure]

5.7 g (75% yield) of the desired compound was obtained by performing the same process as Synthesis Example 1, except that 2′-(8-chlorodibenzo[b,d]thiophen-2-yl)-10-phenyl-10H-spiro[acridine-9,9′-fluorene] of Preparation Example 22 and N-([1,1′-biphenyl]-4-yl)-[1,1′-biphenyl]-2-amine were used.

[LCMS]: 909

### [Synthesis Example 40] Synthesis of Compound 187

[chemical structure]

8.9 g (71% yield) of the desired compound was obtained by performing the same process as Synthesis Example 1, except that 3′-(7-bromo-9,9-dimethyl-9H-fluoren-2-yl)-10,10-dimethyl-10H-spiro[anthracene-9,9′-fluorene] of Preparation Example 23 and N-phenyl-[1,1′-biphenyl]-4-amine were used.

[LCMS]: 794

### [Synthesis Example 41] Synthesis of Compound 198

[chemical structure]

5.8 g (66% yield) of the desired compound was obtained by performing the same process as Synthesis Example 1, except that 2′-(7-bromo-9,9-dimethyl-9H-fluoren-2-yl)-10,10-diphenyl-10H-spiro[anthracene-9,9′-fluorene] of Preparation Example 24 and di([1,1′-biphenyl]-4-yl)amine were used.

[LCMS]: 994

### [Synthesis Example 42] Synthesis of Compound 199

[chemical structure]

7.3 g (68% yield) of the desired compound was obtained by performing the same process as Synthesis Example 1, except that 2′-(7-bromo-9,9-dimethyl-9H-fluoren-2-yl)-10,10-diphenyl-10H-spiro[anthracene-9,9′-fluorene] of Preparation Example 24 and N-([1,1′-biphenyl]-4-yl)-[1,1′-biphenyl]-2-amine were used.

[LCMS]: 994

### [Synthesis Example 43] Synthesis of Compound 212

[chemical structure]

7.7 g (62% yield) of the desired compound was obtained by performing the same process as Synthesis Example 1, except that 4′-(7-bromo-9,9-dimethyl-9H-fluoren-2-yl)-10-phenyl-10H-spiro[acridine-9,9′-fluorene] of Preparation Example 25 and N-phenyl-[1,1′-biphenyl]-4-amine were used.

[LCMS]: 843

### [Examples 1 to 43] Fabrication of Organic Electroluminescent Devices

Compounds 1, 2, 8, 9, 12, 15, 16, 22, 24, 28, 31, 35, 37, 39, 48, 50, 57, 58, 66, 70, 77, 79, 86, 88, 94, 102, 103, 109, 110, 113, 126, 129, 138, 142, 145, 156, 157, 171, 174, 187, 198, 199 and 212 synthesized in the Synthesis Examples were purified to high purity through sublimation according to a conventional known method, and then green organic electroluminescent devices were fabricated using the purified compounds according to the following procedures.

First, a glass substrate having an ITO (indium tin oxide) thin layer coated to a thickness of 1500 Å thereon was ultrasonically washed with distilled water. After washing with distilled water, the coated substrate was ultrasonically washed with solvents, such as isopropyl alcohol, acetone and methanol, dried, transferred into a UV/ozone cleaner (Power sonic 405, Hwashin Tech Co., Ltd.), cleaned with UV for 5 minutes, and then transferred into a vacuum vapor deposition system.

On the ITO transparent glass substrate prepared as described above, m-MTDATA (60 nm), each of compounds 1, 2, 8, 9, 12, 15, 16, 22, 24, 28, 31, 35, 37, 39, 48, 50, 57, 58,66,70,77,79, 86, 88,94, 102, 103, 109, 110, 113, 126, 129, 138, 142, 145, 156, 157, 171, 174, 187, 198, 199 and 212 (80 nm), DS-H522+5% DS-501 (300 nm), BCP (10 nm), Alq₃ (30 nm), LiF (1 nm) and Al (200 nm) were sequentially deposited, thereby fabricating organic EL devices.

DS-H522 and DS-501, used in device fabrication, are the products of Doosan Electronics BG, and m-MTDATA, TCTA, CBP, Ir(ppy)₃ and BCP have the following structures, respectively:

[chemical structure]

### [Comparative Example 1] Fabrication of Organic Electroluminescent Device

An organic EL device was fabricated in the same manner as in Example 1, except that NPB was used as a hole transport layer material instead of Compound 1, which was used as the hole transport layer material when forming the hole transport layer in Example 1. The structure of NPB used is as follows.

[chemical structure]

### Evaluation Example 1

For each of the green organic electroluminescent devices fabricated in Examples 1 to 43 and Comparative Example 1, the driving voltage at a current density of 10 mA/cm², the current efficiency and the emission peak were measured. The results of the measurement are shown in Table 1 below.

TABLE 1

| | | Driving | Current |
|---|---|---|---|
| | Hole transport | voltage | efficiency |
| Sample | layer | (V) | (cd/A) |
| Example 1 | Compound 1 | 3.9 | 24.9 |
| Example 2 | Compound 2 | 4.1 | 25.1 |
| Example 3 | Compound 8 | 4.1 | 23.9 |
| Example 4 | Compound 9 | 4.2 | 25.2 |
| Example 5 | Compound 12 | 4.4 | 22.9 |
| Example 6 | Compound 15 | 3.9 | 24.5 |
| Example 7 | Compound 16 | 3.8 | 24.8 |
| Example 8 | Compound 22 | 4.1 | 23.5 |
| Example 9 | Compound 24 | 4.2 | 23.7 |
| Example 10 | Compound 28 | 3.9 | 22.9 |
| Example 11 | Compound 31 | 4.1 | 24.0 |
| Example 12 | Compound 35 | 3.7 | 22.8 |
| Example 13 | Compound 37 | 3.9 | 22.4 |
| Example 14 | Compound 39 | 4.3 | 25.1 |
| Example 15 | Compound 48 | 4.4 | 24.7 |
| Example 16 | Compound 50 | 4.1 | 24.1 |
| Example 17 | Compound 57 | 4.1 | 24.8 |
| Example 18 | Compound 58 | 3.9 | 20.3 |
| Example 19 | Compound 66 | 4.3 | 19.4 |
| Example 20 | Compound 70 | 4.6 | 21.5 |
| Example 21 | Compound 77 | 4.1 | 24.9 |
| Example 22 | Compound 79 | 4.0 | 25.0 |
| Example 23 | Compound 86 | 4.2 | 25.0 |
| Example 24 | Compound 88 | 4.1 | 24.0 |
| Example 25 | Compound 94 | 3.9 | 22.2 |
| Example 26 | Compound 102 | 4.1 | 22.5 |
| Example 27 | Compound 103 | 4.2 | 25.4 |
| Example 28 | Compound 109 | 4.1 | 24.8 |
| Example 29 | Compound 110 | 4.5 | 21.0 |
| Example 30 | Compound 113 | 4.4 | 24.3 |
| Example 31 | Compound 126 | 3.8 | 24.1 |
| Example 32 | Compound 129 | 4.5 | 21.2 |
| Example 33 | Compound 138 | 3.7 | 24.5 |
| Example 34 | Compound 142 | 4.4 | 23.5 |
| Example 35 | Compound 145 | 4.1 | 23.0 |
| Example 36 | Compound 156 | 4.2 | 24.5 |
| Example 37 | Compound 157 | 4.1 | 24.8 |
| Example 38 | Compound 171 | 3.8 | 24.5 |
| Example 39 | Compound 174 | 3.9 | 25.2 |
| Example 40 | Compound 187 | 4.4 | 23.1 |
| Example 41 | Compound 198 | 4.5 | 21.3 |
| Example 42 | Compound 199 | 4.8 | 24.9 |
| Example 43 | Compound 212 | 4.2 | 24.7 |
| Comparative | NPB | 5.2 | 18.2 |
| Example 1 | | | |

As shown in Table 1 above, it can be seen that the organic electroluminescent device (the organic electroluminescent device fabricated in each of Examples 1 to 43) using the compound of the present disclosure as a hole transport layer exhibited higher current efficiency and a lower driving voltage compared to the case in which conventional NBP was applied (Comparative Example 1).

1. A compound represented by the following Formula 1: [chemical structure]
wherein
X is N, O, S or C;
L is a direct bond or is selected from the group consisting of C₆-C₁₈ arylene group and a heteroarylene group having 5 to 18 nuclear atoms;
Ar₁ and Ar₂ are each independently selected from the group consisting of hydrogen, deuterium, halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄₀ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylamine group, and are symmetrical or asymmetrical with respect to each other; and
the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of Ar₁ and Ar₂ are each independently unsubstituted or substituted with one or more substituents selected from the group consisting of deuterium, halogen, a cyano group, a nitro group, a C₁-C₄₀ alkyl group, a C₂-C₄₀ alkenyl group, a C₂-C₄₀ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents are the same or different.

2. A compound represented by the following Formula 2: [chemical structure]
wherein
X, L, Ar₁ and Ar₂ are as defined in claim 1;
R₁ and R₂ are each independently selected from the group consisting of hydrogen, deuterium, halogen, a cyano group, a nitro group, a C₁-C₄₀ alkyl group, a C₂-C₄₀ alkenyl group, a C₂-C₄₀ alkynyl group, a C₃-C₄₀ cycloalkyl group, a heterocycloalkyl group having 3 to 40 nuclear atoms, a C₆-C₆₀ aryl group, a heteroaryl group having 5 to 60 nuclear atoms, a C₁-C₄ alkyloxy group, a C₆-C₆₀ aryloxy group, a C₃-C₄₀ alkylsilyl group, a C₆-C₆₀ arylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and C₆-C₆₀ arylamine group; and
the alkyl group, alkenyl group, alkynyl group, aryl group, heteroaryl group, aryloxy group, alkyloxy group, cycloalkyl group, heterocycloalkyl group, arylamine group, alkylsilyl group, alkylboron group, arylboron group, arylphosphanyl group, mono- or diarylphosphinyl group and arylsilyl group of R₁ and R₂ are each independently unsubstituted or substituted with one or more substituents selected from the group consisting of deuterium, halogen, a cyano group, a nitro group, a C₁-C₄₀ alkyl group, a C₂-C₄₀ alkenyl group, a C₂-C₀₄ alkynyl group, a C₆-C₆₀ aryl group, a heteroaryl group having 5 to 60 nuclear atoms, a C₆-C₆₀ aryloxy group, a C₁-C₄₀ alkyloxy group, a C₆-C₆₀ arylamine group, a C₃-C₄₀ cycloalkyl group, a heterocycloalkyl group having 3 to 40 nuclear atoms, a C₁-C₄₀ alkylsilyl group, a C₁-C₄₀ alkylboron group, a C₆-C₆₀ arylboron group, a C₆-C₆₀ arylphosphanyl group, a C₆-C₆₀ mono- or diarylphosphinyl group, and a C₆-C₆₀ arylsilyl group, and when they are substituted with a plurality of substituents, these substituents are the same or different.

3. The compound of claim 1, wherein
R₁ and R₂ are each independently selected from the group consisting of a C₁-C₄₀ alkyl group, a C₆-C₆₀ aryl group, and a heteroaryl group having 5 to 60 nuclear atoms, and
the alkyl group, aryl group and heteroaryl group of R₁ and R₂ are each independently unsubstituted or substituted with one or more substituents selected from the group consisting of a C₁-C₄₀ alkyl group, a C₆-C₆₀ aryl group, and a heteroaryl group having 5 to 60 nuclear atoms, and when they are substituted with a plurality of substituents, these substituents are the same or different.

4. The compound of claim 1, wherein
L is a direct bond or is one or more linkers selected from the group consisting of the following Formulas 3 to 8:

[chemical structure]

5. The compound of claim 1, wherein
Ar₁ and Ar₂ are selected from the group consisting of a C₁-C₄₀ alkyl group, a C₆-C₆₀ aryl group, and a heteroaryl group having 5 to 60 nuclear atoms, and
the alkyl group, aryl group and heteroaryl group of Ar₁ and Ar₂ are each independently unsubstituted or substituted with one or more substituents selected from the group consisting of a C₁-C₄₀ alkyl group, a C₆-C₆₀ aryl group, and a heteroaryl group having 5 to 60 nuclear atoms, and when they are substituted with a plurality of substituents, these substituents are the same or different.

6. The compound of claim 1, wherein the compound is represented by the following formula:

[chemical structure]

7. The compound of claim 1, wherein the compound is selected from the group consisting of the following compounds:

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

8. An organic electroluminescent device comprising: (i) an anode, (ii) a cathode, and (iii) one or more organic layers interposed between the anode and the cathode,
wherein at least one of the one or more organic layers comprises the compound of claim 1.

9. The organic electroluminescent device of claim 8, wherein the organic layer is one or more selected from among a hole injection layer, a hole transport layer, an auxiliary hole transport layer, an electron injection layer, an electron transport layer, an auxiliary electron transport layer, and a light-emitting layer.

10. The compound of claim 2, wherein
R₁ and R₂ are each independently selected from the group consisting of a C₁-C₄₀ alkyl group, a C₆-C₆₀ aryl group, and a heteroaryl group having 5 to 60 nuclear atoms, and
the alkyl group, aryl group and heteroaryl group of R₁ and R₂ are each independently unsubstituted or substituted with one or more substituents selected from the group consisting of a C₁-C₄₀ alkyl group, a C₆-C₆₀ aryl group, and a heteroaryl group having 5 to 60 nuclear atoms, and when they are substituted with a plurality of substituents, these substituents are the same or different.

11. The compound of claim 2, wherein
L is a direct bond or is one or more linkers selected from the group consisting of the following Formulas 3 to 8:

[chemical structure]

12. The compound of claim 2, wherein
Ar₁ and Ar₂ are selected from the group consisting of a C₁-C₄₀ alkyl group, a C₆-C₆₀ aryl group, and a heteroaryl group having 5 to 60 nuclear atoms, and
the alkyl group, aryl group and heteroaryl group of Ar₁ and Ar₂ are each independently unsubstituted or substituted with one or more substituents selected from the group consisting of a C₁-C₄₀ alkyl group, a C₆-C₆₀ aryl group, and a heteroaryl group having 5 to 60 nuclear atoms, and when they are substituted with a plurality of substituents, these substituents are the same or different.

13. The compound of claim 2, wherein the compound is represented by the following formula:

[chemical structure]

14. The compound of claim 2, wherein the compound is selected from the group consisting of the following compounds:

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

15. An organic electroluminescent device comprising: (i) an anode, (ii) a cathode, and (iii) one or more organic layers interposed between the anode and the cathode,
wherein at least one of the one or more organic layers comprises the compound of claim 2.

16. The organic electroluminescent device of claim 15, wherein the organic layer is one or more selected from among a hole injection layer, a hole transport layer, an auxiliary hole transport layer, an electron injection layer, an electron transport layer, an auxiliary electron transport layer, and a light-emitting layer.
